(19) United States
(12) Patent Application Publication
(10) Pub. No.: US 2006/0052023 A1
(43) Pub. Date: Mar. 9, 2006

(54) Nonwoven material with elastic properties, related production method and device therefor

(76) Inventors: Keld Lauridsen, Aalborg SO (DK); Mette Due, Gistrup (DK); Jorgen Bech Madsen, Aalborg (DK); Thomas Broch-Nielsen, Klarup (DK)

Correspondence Address: Dilworth & Barrese, 333 Earle Ovington Blvd, Suite 702, Uniondale, NY, 11553, US

(21) Appl. No.: 10/532,629

(22) PCT Filed: Oct. 22, 2003
(86) PCT No.: PCT/EP03/11706

§ 371 Date: Apr. 25, 2005

(30) Foreign Application Priority Data
Oct. 23, 2002 (DE) 102 49 431.2

(51) Int. Cl. B32B 5/06; D04H 1/56; B32B 5/26; D04H 3/16

(52) U.S. Cl. 442/389; 442/328; 442/329; 442/361; 442/382; 442/400; 442/401

(57) ABSTRACT

The invention relates to a nonwoven material having elastic properties aligned in one direction, consisting of either one multilayer composite including at least one layer in which fibers or filaments made of an elastic polymer are contained, or of a homogeneous fiber and filament mix in which a portion of the fibers consists of an elastic polymer, wherein in each case a larger part of the fibers or filaments is aligned in a direction under the application of heat which extends transversely to the direction in which the nonwoven material is elastic. The invention furthermore relates to a method for the manufacture of the nonwoven material and to an apparatus to carry out the method.

The invention relates to a nonwoven material having elastic properties.

The term “fibers” used within the framework of this invention relates both to staple fibers and to continuous fibers (filaments).

Due to their versatile application and the unique product properties which can be achieved, nonwoven materials are widespread in the most varied application areas today. For instance, nonwoven materials are used in the area of hygiene products, medical products, protective clothing, cleaning tissues, packaging materials, depths filters, automobile fitting materials, construction materials and in many other areas. The function of the nonwoven materials in this use can be defined as follows:
protective and barrier function;
liquid transport and absorption properties;
filtration, separation or retention of particles;
reinforcement.

One of the main disadvantages of the nonwoven materials in the prior art, for example of needlepunched or spunlace, spunbonded or spunmelted nonwovens, is that they do not have any elasticity or stretchiness or have them only to a very limited degree. The problem furthermore exists that nonwoven materials of the prior art, for example spunmelted composite products, lose their material properties, for example the liquid barrier function, on stretching of the material.

Increasing demands and needs of consumers and market requirements derived from this result in new demands on the nonwoven materials, with following key parameters being important:
new, consumer-focused properties;
higher performance capability and increased comfort with lower costs;
product flexibility for easier adaptation to fast-changing market trends and product designs;
constant product quality;
economic manufacturing methods for the provision of nonwoven materials.

To satisfy market demands, it is necessary to provide nonwoven materials provided with elastic properties in the most varied areas, for example to improve the properties of diapers, personal care products for women, protective mats, poster materials and similar, where it is a question of providing an improved fit, with the other positive properties having to be maintained.

Various attempts have already been made to provide nonwoven materials with elastic properties. However, solutions only resulted which are very complex and were thus expensive and which were inadequate with respect to comfort and to the barrier properties. For example materials having elastomeric properties were thus worked into the nonwoven materials, with the elasticity being produced by a combination of the nonwoven material with elastic stretching material or elastic bands made of natural or synthetic rubber.

Disposable products consisting of the aforesaid nonwoven materials only have low spread experience since they are comparatively expensive.

Another attempt to produce elastic properties in nonwoven materials results, for example, from the U.S. Reissue Pat. No. 35,206 in which composite materials consisting of non-elastomeric fibers are stretched under heat to reduce the pore size for use in filtration processes. This material has a poor recovery property after a corresponding stretching or an overall low stretchability.

Polyurethane foam has, for example, been used in the prior art or an elastic film material has been combined with the nonwoven material. Another prior art is known from U.S. Pat. No. 5,5851,935 which relates to a laminated elastomeric material which is elastic in cross-section. This laminate contains an elastomeric film having one or two layers of nonwoven material which consists of carded thermoplastic stable fibers and is spot bonded thereto. The use of specific polystyrene copolymers in individual meltblown layers, such as resulted for example from U.S. Pat. No. 5,324,580, had already been presented as another possibility.

All nonwoven materials already known, such as needlepunched, spunlaced, spunbond or spunmelt products, suffered from the disadvantage that they only have a slow recovery property, elasticity and stretchability. A whole series of previously known nonwoven materials such as spunmelt composite products moreover lose their functional properties such as the liquid barrier property and the recovery property if they are stretched during use.

Elastic films have a low breathability, or none at all, very differently to nonwoven materials. Where foam was used in the prior art, there is no breathability at all.

The composite materials in accordance with the prior art were manufactured by relatively complex offline solutions in that the starting nonwoven materials were bonded to the elastic film layers or the elastic foam offline.

Due to their structure, the meltblown nonwoven materials in accordance with the prior art only have a low strength and wear resistance. Conventional polypropylene meltblown nonwovens are furthermore very brittle, this means that they have no elasticity, which has the result that their barrier properties fall greatly on corresponding stretching during their use.

The industrial use of meltblown nonwovens is only reduced to niche applications due to these disadvantages.

Nonwoven laminates made of elastic net fabrics, elongated yarns/filaments or woven structures can be named as further elastic materials. These laminates are comparatively expensive and do not permit any homogeneous material processing.

It is now the object of the present invention to provide a nonwoven material which has elastic properties, on the one hand, for instance a very high stretchability and a very good recovery property. On the other hand, the usual advantages of nonwoven materials, namely the breathability, the barrier property and the tensile strength should be maintained. In this connection, in particular the liquid barrier property, but also the particle retention property, is to be understood by barrier property. Furthermore, an improved wearing feeling and touching properties, comfort, good opaqueness and a homogeneous textile can be achieved at low cost without the disadvantages of laminates.

In accordance with the invention, this object is solved by the feature combination of claim 1.

Here, a nonwoven material is proposed which has elastic properties aligned in one direction and consists either of a multilayer composite comprising at least one layer in which fibers or filaments of an elastic polymer are contained or of a homogeneous fiber and filament mix in which some of the fibers consist of an elastic polymer. In addition, a respective larger part of the fibers or filaments is aligned under the application of heat in a direction extending transversely to the direction in which the nonwoven material is elastic. The share of elastic polymer advantageously amounts to at least 10% by weight. The good elasticity properties and excellent recovery properties of the material can be achieved by the combination of the selected materials with the alignment of most fibers or filaments in one direction under the application of heat. The barrier functions, which were achieved by the manufacture of microfibers or microfilaments provided with elastic properties, can particularly advantageously also be maintained during the use of the materials, that is with correspondingly frequent stretching.

Preferred aspects of the invention result from the dependent claims following on from the main claim.

The multilayer composite can accordingly contain elastic meltblown fibers and spunbond fibers.

The elastic meltblown fibers can comprise bicomponent fibers with an elastic portion. The added spunbond fibers do not necessarily have to be elastic.

The homogeneous fiber mix can consist of a needled felt and/or of a spunlaced product in which elastic fibers have been added.

A homogeneous fiber mix of a needled felt and/or of a spunlaced product can be combined with at least one layer of elastic meltblown fibers and/or spunbond fibers.

The composite and the needled felt and the spunlaced product can also contain viscose or natural fibers such as cellulose in addition to synthetic fibers.

One or more meltblown layers (M) can be arranged between one or more spunbond layers (S), for example in the order SM, SMS, SMMS, SSMMS, SSMMSS, with the elastomeric layers being contained at least in one meltblown layer.

The elastic nonwoven layer can be a liquid barrier—or a particle retention layer.

The properties as a liquid barrier layer or a particle retention layer can also be maintained after straining or stretching of the nonwoven material.

The product stretchability can amount to up to 700%, preferably 50-400%. The recovery property, which is also designated as the recovery in English, can amount to at least 60% on a two-fold drawing by 100%. On a two-fold drawing by 150%, it can amount to at least 50%. The preferred range of the recovery property lies at least at 80% on a two-fold drawing by 100% and at least at 70% on a two-fold drawing by 150%.

The nonwoven material in accordance with the invention is preferably breathable and hydrophobic.

The treatment with a hydrophilic coating material, for example with a surface-active agent, or with additives results in hydrophilic properties of the nonwoven such as moisture absorption and fluid transport.

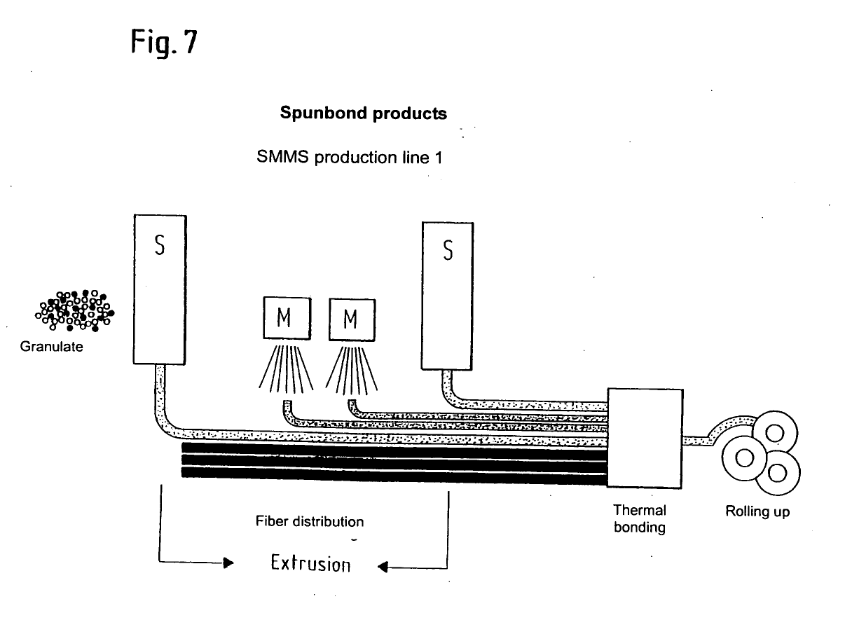

If polymers with elastic properties are used as meltblown fibers, they should preferably have similar flow properties with respect to the rheological properties and viscosity properties as polypropylene. Such a material can preferably be manufactured on the fabrication machines for conventional nonwoven materials (FIG. 7), which consist of polypropylene, for example. The material is preferably manufacturable on an industrial production plant with high productivity, for example on Reicofil plants.

In accordance with a particular aspect of the invention, the meltblown fibers can consist of the following mixture: more than 60% by weight of a triblock copolymer consisting of 70% by weight styrene-ethylene/butylene-styrene and 30% by weight styrene-ethylene/butylene, where the polystyrene share of the polymer is 14% by weight (e.g. Kragon G®), 5-35% by weight polypropylene, which is suitable for processing in the meltblown processes, and an anti-blocking agent to improve the flow properties. Mixtures without anti-blocking agents, e.g. consisting of 75% Kraton G and 25% MFR 800 PP, have a reduced processing capability on the use of meltblown equipment, which is due to the reduced flow properties and thus to the reduced performance of the extruder and of the nozzle.

The meltblown fibers can also consist of an elastic polyolefin, for example of a metallocene-catalyzed copolymer of the polyethylene and/or polypropylene.

The meltblown fibers can also consist of a thermoplastic elastic polyurethane.

With a multilayer design, in addition to at least one meltblown layer with elastic fibers, spunbond layers made of one of the following materials can be present: of polyolefin or polyester, or bicomponent polymer based on polypropylene and polyethylene, or of a polypropylene or polyester mixed with a bicomponent polypropylene/polyethylene, or of an elastic polymer such as a polyurethane, polystyrene block copolymer or an elastic polypropylene and/or polypropylene.

The spunbond layers and/or meltblown layers can have a different structure within the framework of the invention.

The individual layers of the multilayer design can be bonded to one another by needlepunching, spunlacing, by thermobonding, by calendering with smooth rolls and/or engraved rolls and/or infrared bonding.

The basis weight of the multilayer design can amount to between 7 g/m³ to 400 g/m³, with the elastic meltblown layers amounting to 1 to 60% by weight.

The basis weight of the needlepunched nonwoven/spunlaced product or needlepunched nonwoven as a multilayer design together with elastic meltblown layers can amount to 40-700 g/m³, with the elastic meltblown layers amounting to 1 to 60% by weight.

The meltblown layer provided with elastic properties can have a fiber thickness of 0.01 to 1.2 denier, preferably 0.01 to 0.5 denier.

A further part of the invention consists of a method of manufacturing one of the aforesaid nonwoven materials. After manufacture of a nonwoven material from one of the materials described above, the method in accordance with the invention now consists of drawing the prefabricated nonwoven material web either in the running direction or transversely to the running direction for the alignment of the fibers or filaments under the application of heat. A respective elasticity in a direction perpendicular to the drawing direction is generated by the corresponding drawing under the application of heat and by the alignment of the fibers and filaments thus achieved.

To generate the elastic properties of the nonwoven material in the longitudinal direction and the increase of the basis weight belonging thereto, the transport speed can be lowered more, measured in %, in the longitudinal direction than the width increase in %. The nonwoven material web is hereby widened, whereby elastic properties result in the longitudinal direction and overall at the increase of the basis weight. To generate the elastic properties of the nonwoven material in the transverse direction and the increase of the basis weight belonging thereto, it is thereby generated that the width restriction, measured in %, is higher than the transport speed in the longitudinal direction.

An apparatus in accordance with the invention to carry out the aforesaid method comprises an oven and at least one drawing device to draw the nonwoven material web.

The drawing device in this process can have two wheel-shaped gripping apparatuses arranged to the side of the nonwoven material web and having receiving regions arranged at their periphery to grip the nonwoven material web for the drawing of the nonwoven material web in the direction transverse to its transport direction.

The drawing device can preferably consist of at least two oppositely disposed rolls to draw the nonwoven material web in the direction longitudinal to its transport direction, the nonwoven material web being fixed by friction by said rolls and being pulled at a higher speed compared to the entry speed of the nonwoven material web into the oven so that the nonwoven material web is pulled in the longitudinal direction.

A temperature is advantageously set in the apparatus inside the oven between the softening point and the melting point of the respectively processed thermoplastic fibers.

The processing speed of the nonwoven material web amounts to 5 to 150 m/min on drawing in the width and to 5 to 400 m/min on drawing in the longitudinal direction.

The particular advantage of the present invention consists of the fact that here nonwoven materials are provided whose properties can be tailored to the respective individual demands. These properties consist of the good recovery property after a corresponding stretching, the high stretchability, the liquid barrier function, the breathability of the respectively functional performance and the comparatively low manufacturing costs. The following examples can be given in this connection.

A first example consists of an elastic breathable nonwoven material having a textile surface and a liquid barrier function. The product weight, the elasticity, the recovery property, the strength and the barrier function can be set such that the material can be used as a leg collar or as a stomach band in diapers or in protective clothing. The nonwoven material can be a composite material in which the elastic material should be part of the barrier layer. It is achieved by the use of microfibers put into an elastic state which are present either as meltblown fibers or as bicomponent split fibers as part of the barrier layer. Another application can consist of the fact of substituting a following film by the material in accordance with the invention or of accordingly substituting at least part of the film, for example on use in hygiene products, to achieve barrier properties here and good elasticity with better comfort. A preferred use in particular in the field of diapers thus results.

Due to the excellent elastic properties of the nonwoven material, it can, however, also be used in the furniture industry as a cover material or as a bed covering material. The elasticity of the material increases the comfort here and facilitates the handling of the material. For instance, handling capability can be substantially facilitated on a corresponding covering of pieces of furniture or of bed mattresses, since the elastic material lies easily at the corners and edges of the respective piece of furniture or of the mattresses. In this application case, the nonwoven material can consist of a composite material in which the material provided with elastic properties is combined with other nonwoven materials in order to achieve improved physical properties, for example an improved strength and an improved visual appearance. A resiliently porous elastic nonwoven material with stretch properties can be used as a substitute for foam material in an application in the field of upholstery manufacture and cushion manufacture with respect to its, product weight, the elasticity, the strength and a possible barrier function.

The elastic nonwoven material can, if desired, be treated such that it becomes hydrophile on one or both sides or has hydrophilic or hydrophobic zones. In this process, the product weight, elasticity, recovery property, strength and hydrophilic properties can be adapted such that the material can be used as a clothing material or as a cover material. The material here in particular has good comfort in wear and a good fitting shape.

The aforesaid advantageous application areas are only listed by way of example and can be supplemented by any other examples in which the advantageous product properties of the material in accordance with the invention come to bear.

Further details and advantages of the invention result from the embodiments explained in the following with reference to the drawing. There are shown:

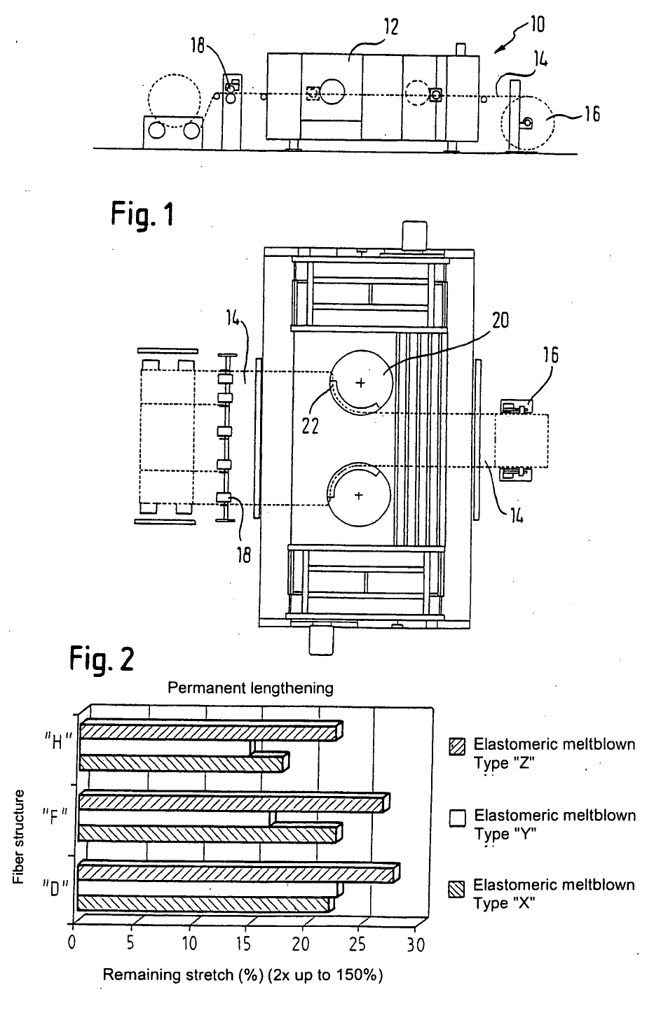

FIGS. 1a, b: a schematic side view and a plan view of part of an apparatus in accordance with the invention for the manufacture of the nonwoven material in accordance with the invention;

FIG. 2: a diagram to illustrate the permanently remaining lengthening of the material, also in dependence on the share of the meltblown fibers in % by weight and in the elastomeric share;

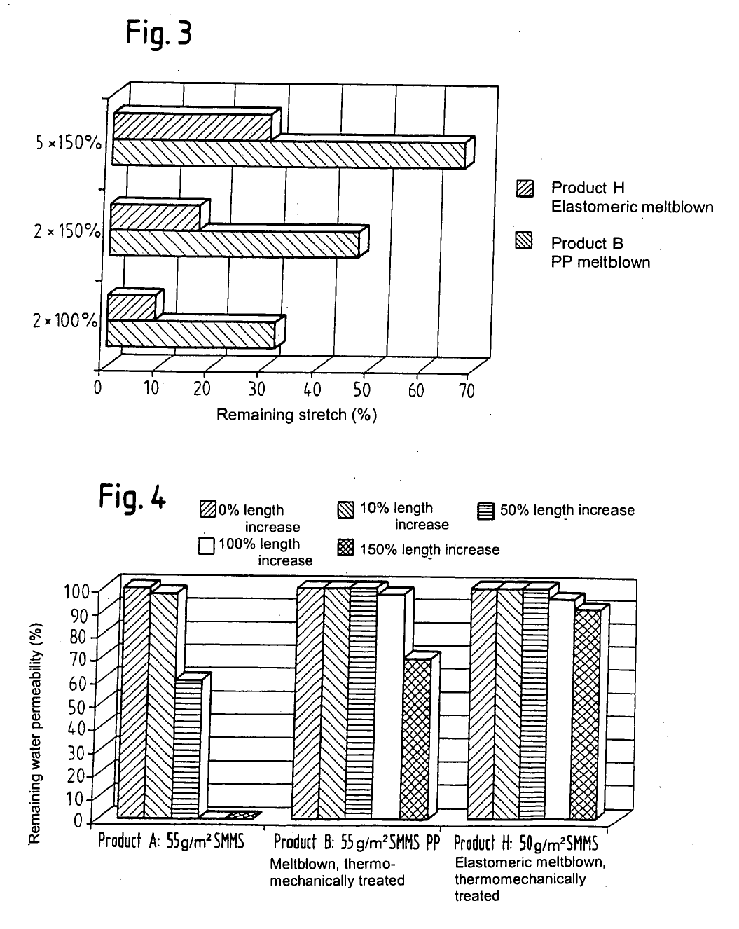

FIG. 3: permanently remaining lengthening on different longitudinal stretching procedures and different stretching cycles;

FIG. 4: a diagram to illustrate the barrier properties in the stretched state of the material in accordance with the invention;

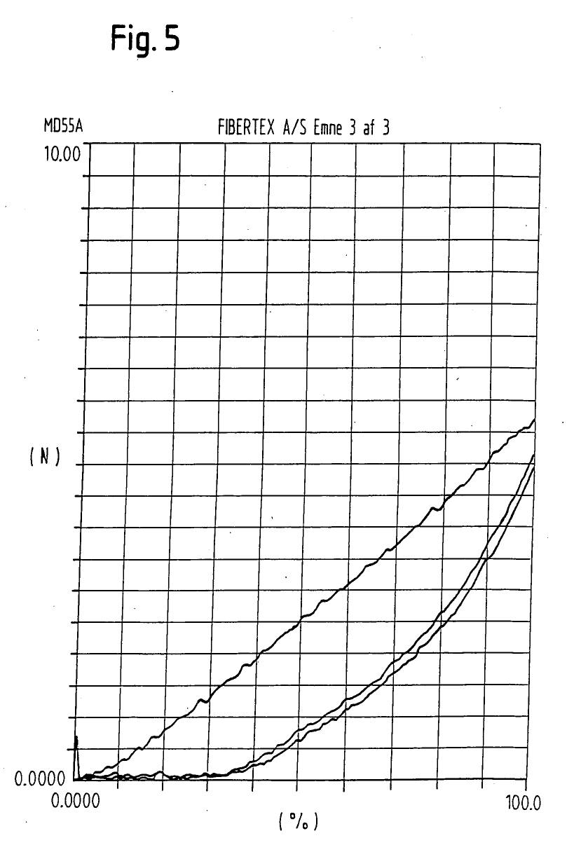

FIG. 5: a stretch-test diagram in which an SMMS material having meltblown fibers in accordance with the prior art is used;

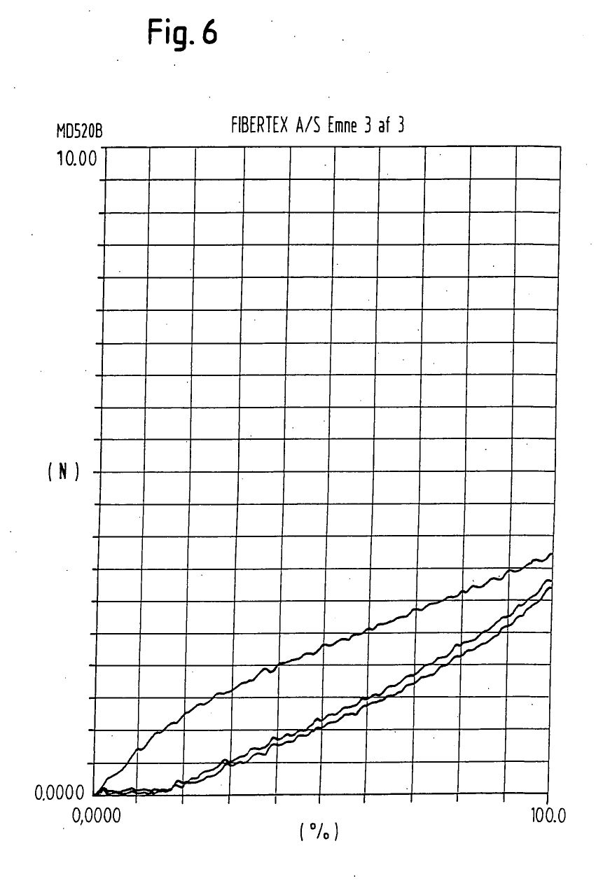

FIG. 6: a stretch-test diagram in which an SMMS material in accordance with the prior art containing elastomeric meltblown fibers is tested;

FIG. 7: a schematic illustration of the manufacture of SMMS material.

An apparatus is shown in FIG. 1 in which the starting nonwoven materials, which come from a production machine known per se, are further processed such that their fibers or filaments are preferably oriented in one direction. With this apparatus, a stretching in the direction transverse to the conveying direction of the nonwoven material web can be generated, on the one hand so that here an elastic property is achieved in the longitudinal direction of the nonwoven material web. Alternatively, elasticity can be generated in the transverse direction of the nonwoven material web by a corresponding stretching in the longitudinal direction of the nonwoven material web.

The core of the apparatus 10 consists of an oven 12 through which the nonwoven material web 14 is guided. The nonwoven material web 14 is removed from a correspondingly supported roll 16. The nonwoven material web 14 is pulled forward by a feed roll pair 18 between which the nonwoven material web 14 is clamped. Wheel-shaped gripping apparatuses 20 with reception regions to grip the nonwoven material web 22 arranged at their periphery are arranged inside the oven space to the side of the nonwoven material web. These reception regions arranged at their periphery are here only shown in part of the periphery of the wheel-shaped gripping apparatuses 20 in FIG. 1. However, they run around the whole periphery of the wheel-shaped gripping apparatuses. The nonwoven material web is taken up by means of these reception regions and, as shown in FIG. 1 b, is stretched laterally, that is is essentially widened. To now generate elasticity in the longitudinal direction of the nonwoven material web, the speed of the nonwoven material web in the longitudinal direction is lowered such that a drawing in the width becomes possible. In this process, the material is drawn in the width faster here than it is moved on in the longitudinal direction so that the total nonwoven material web becomes wider as a result and has a higher basis weight.

During the stretching in the width, the nonwoven material web 14 is heated up so far inside the oven 12 that the temperature lies between the softening point and the melting point of the respective thermoplastic fiber material. The respectively used wheel-shaped gripping apparatuses can be selected in their diameter in dependence on the desired stretching of the nonwoven material web. The stretching rate for the nonwoven material web usually lies between 5% and 500%.

If elasticity transversely to the longitudinal direction of the nonwoven material web should be generated with the apparatus shown in FIG. 1, the wheel-shaped gripping apparatuses 20 are not used. In this case, the nonwoven material web 14 is drawn in longitudinal stretching during the heating in the oven 12, with the roll pairs 18—between which the nonwoven material web is clamped—being driven at a speed which is higher than the entry speed of the nonwoven material web 14 into the oven 12. The nonwoven material web is given an elasticity in the transverse direction by this longitudinally directed stretching process. The fibers and filaments are predominantly aligned in the longitudinal direction in this process. Since the nonwoven material web 14 is not fixed at the side, its width is reduced in the direction transverse to the propagation direction of the nonwoven material web.

The products improved in their elastic properties using the apparatus shown in accordance with FIG. 1 were examined as to their properties, with different nonwoven materials being used here to be able to adjust the stretch property, the recovery property and the barrier functions of the respective nonwoven material web.

The tensile strength on tearing and the lengthening under the application of different loads is measured in accordance with ERT 20.2/89 with respect to determining the elasticity.

The recovery property is determined in that the nonwoven material is stretched to a pre-determined length stretching for a pre-determined number of load cycles and is respectively relaxed for two minutes before the permanently remaining lengthening of the nonwoven material web is measured.

The watertightness of the product, expressed as a hydrostatic head, is used as the barrier function. This measurement was carried out in accordance with the standard ERT 120.1/80.

In the Table 1 shown below, product information on the nonwoven materials used in the trials is reproduced. The spunbond fibers are all made from polypropylene (with the exception of product P in which metallocene propylene was used). The needle felt product is manufactured from polypropylene staple fibers. P designates the starting nonwoven material and O the heat-treated nonwoven material in which the larger part of the fibers is aligned in one direction. The statement of base weight relates to the respective starting nonwoven material.

TABLE 1

| | | | Meltblown | Process | |
|---|---|---|---|---|---|
| | | Design | Elastomer | | Temp. |
| Product | Type | S/M/M/S (g) | (%) | V9/V8 | (° C.) |
| A (P) | SMMS | 20/7.5/7.5/20 | 0 | — | — |
| B (O) | SMMS | 20/7.5/7.5/20 | 0 | 1.5 | 140 |
| C (P) | SMMS | 17.5/2.5/2.5/17.5 | 70 | — | — |
| D (O) | SMMS | 22.5/2.5/2.5/22.5 | 70 | 1.4 | 135 |
| E (P) | SMMS | 20/5/5/20 | 70 | — | — |
| F (O) | SMMS | 20/5/5/20 | 70 | 1.4 | 135 |
| G (P) | SMMS | 17.5/7.5/7.5/17.5 | 70 | — | — |
| H (O) | SMMS | 17.5/7.5/7.5/17.5 | 70 | 1.4 | 135 |
| I (O) | Needle | — | 0 | 1.4 | 141 |
| | felt | | | | |
| J (O) | SMMS | 4/1/1/4 | 70 | 1.25 | 135 |
| | Phi | | | | |
| K (O) | SMMS | 5/1.5/1.5/5 | 70 | 1.25 | 135 |
| L (O) | SMMS | 6/1.5/1.5/6 | 70 | 1.3 | 135 |
| M (O) | SMMS | 7/1.5/1.5/7 | 70 | 1.3 | 135 |
| N (O) | SMMS | 8.5/1.5/1.5/8.5 | 70 | 1.3 | 135 |
| O | MM | 7.5/7.5 | 70 | — | — |
| P (O) | SMMS | 6/1.5/1.5/6 | — | 1.25 | 137 |
| | (MPP) | | | | |

The materials are thermomechanically modified since a large part of the fibers is aligned in one direction. Excellent stretching properties, recovery properties and barrier properties hereby result. These particularly good properties result from the representation in accordance with Table 2. Product B shows the properties of a product in accordance with the prior art, whereas the products D, F and H contain elastomeric meltblown fibers and have a substantially improved stretching property. In Table 2, the stretching property of the respectively collectively treated nonwoven materials is shown in which a large part of the fibers is aligned in one direction. The weight values relate to the non-heat treated nonwoven material.

TABLE 2

| | | Product B | Product D | Product F | Product H | Product I |
|---|---|---|---|---|---|---|
| | | (O) | (O) | (O) | (O) | (O) |
| | | SMMS | SMMS | SMMS | SMMS | Needle felt |
| | Unit | 55 gsm | 50 gsm | 50 gsm | 50 gsm | 80 gsm |
| Tensile | N/5 cm | 10 | 17 | 16 | 15 | 50 |
| strength | | | | | | |
| Length | % | 200-250 | 330-380 | 350-400 | 400-450 | 200-250 |
| stretching | | | | | | |
| on tearing | | | | | | |
| Elastomers | — | No | Yes | Yes | Yes | No |
| contained | | | | | | |

In Table 3, data are shown in addition to the stretching property with respect to the recovery property and the barrier function of products with a low basis weight which contain elastomeric meltblown fibers. Here, too, the weight data refer in each case to the non-heat treated starting nonwoven material.

TABLE 3

| | | | | | | Product P |
|---|---|---|---|---|---|---|
| | Product J | Product K | Product L | Product M | Product N | (O) |
| | (O) | (O) | (O) | (O) | (O) | 15 gsm |
| | 10 gsm | 13 gsm | 15 gsm | 17 gsm | 20 gsm | SMMS |
| | SMMS Hi | SMMS | SMMS | SMMS | SMMS | MPP |
| Stretch | 6 | 8 | 10 | 12 | 16 | 15 |
| strength on | | | | | | |
| tearing | | | | | | |
| (N/5 cm) | | | | | | |
| Lengthening | 200-400 | 200-40 | 200-400 | 200-400 | 200-400 | 400-600 |
| on tearing | | | | | | |
| (%) | | | | | | |
| Air permeability | 750 | 800 | 700 | 650 | 530 | 500 |
| (l/m²/s) | | | | | | |
| Recovery | 5-10 | 5-10 | 5-10 | 5-10 | 5-10 | 5-10 |
| (%) | | | | | | |
| 2 × to 100% | | | | | | |

The product J has a very low basis weight (10 g/m²), on the one hand. Nevertheless, this hydrophilic SMMS nonwoven material has a defined pore size distribution. The product concept using very light spunmelt composite products combines particularly good hydrophilic properties with a good particle retention property so that overall an improved SAP barrier property is achieved. At the same time, a softer product has been provided due to the fine meltblown fibers and spunbond fibers.

The products with a basis weight of 13-20 g/m² (the products K, L, M, N and P) are suitable for applications in which a soft textile surface, good recovery properties, a good stretching property and a barrier function are required. The use in diapers suggests itself here, for example as a stomach band or as a leg collar. A use as protective clothing is also preferably possible with this product. In particular the maintenance of the barrier property during the stretching characterizes this material over the known materials.

The product which consists of the spunbond fibers using metallocene polypropylene shows extremely high stretching properties.

The product O, a meltblown nonwoven material which consists of elastomeric components, shows the following properties:

TABLE 4

| | Unit | Product O |
|---|---|---|
| Base weight | G/m² | 15 |
| Tensile strength | N/5cm 1.5 | 1.5 |
| Stretch at breaking point | % | 500-700 |
| Remaining deformation (2x | % | 7 |
| at 150%) | | |
| Fiber thickness | Denier | 0.03-0.6 |
| Air permeability | L/m²/s | 600-900 |

A base weight of 15 g/m², at a stretching strength of 1.5 N/5 cm. The longitudinal stretching on tearing amounts to 500 to 700% and a permanent longitudinal stretching at two-fold stretching to 150% amounts to only 7%. The fiber thickness amounts to 0.03 to 0.6 denier and the air permeability amounts to 600 to 900 L/m²/s. The fiber thickness within the elastomeric meltblown fiber layers amounts to 0.01 denier to 1 denier, but should preferably lie between 0.01 and 0.05 denier to have the best possible barrier function and recovery properties.

Depending on the elastomeric component used (for example a Kraton®) composite or an elastomeric polyolefine), which is used for the meltblown layer, it is possible to match the recovery properties of the product to the respective demand. As results from FIG. 2 and the Table 5 reproduced in the following, the recovery property is dependent to a great extent on the type of the elastomeric material used and naturally on its proportionate amount. The corresponding properties in accordance with the present invention are substantially improved in comparison with the recovery properties of SMMS materials in accordance with the prior art. In the prior art, for example in U.S. Pat. No. 35,206, a recovery property of an SMMS material of 60% is achieved with a 50% lengthening, which means that a permanent lengthening of 40% is achieved after an interplay. The products in accordance with the present invention, however, in comparison, reach a recovery property of more than 70% on a two-fold lengthening by 150%.

TABLE 5

| | Remaining lengthening (2x to 150%) | | |
|---|---|---|---|
| | “D” SMMS | | |
| | (22.5/2.5/ | “F” SMMS | “H” SMMS |
| | 2.5/22.5) | (20/5/5/20) | 17.5/7.5/7.5/17.5) |
| Elastomeric meltdown | 22% | 22.5% | 17.7% |
| Type A | | | |
| Elastomeric meltdown | 22.7% | 16.7% | 1.7% |
| Type B | | | |
| Elastomeric meltdown | 27.7% | 26.7% | 22.3% |
| Type C | | | |

In Table 5, the remaining lengthening of thermomechanically treated SMMS material having elastomeric meltblown fibers is shown. These products are stretched two-fold by 150%.

In FIG. 2, the permanent lengthening of the material web is established in dependence on the selected product (cf. the values in Table 5).

It can be found as a result that the materials having elastomeric meltblown fibers have a long-term recovery property. Even after five movement cycles in which a length stretching of 150% takes place, a recovery property of 70% still results, as results from FIG. 3. In FIG. 3, the change cycles of the lengthening of the web are varied.

In the Table 6 reproduced below, the air permeability of the product is shown in the stretches state for an SMMS product having a basis weight of 50 g/m² having elastomeric meltblown fibers.

TABLE 6

| | Air permeability (l/m²/s in the stretched state | | | | | | | |
|---|---|---|---|---|---|---|---|---|
| Product | 0% | 10% | 20% | 30% | 40% | 50% | 100% | 150% |

| | | | | | | | | |
|---|---|---|---|---|---|---|---|---|
| H (O) | 350 | 550 | 750 | 900 | 1200 | 1350 | 1800 | 1950 |

Products which have an elastomeric meltblown portion show a large and obvious increase in the air permeability and breathability in the stretched state. However, the barrier layer provided with elastic properties has the result that the water impermeability of the product remains maintained, while the product is stretched. Corresponding data result from the following table 7 and from the FIG. 4 in which the water permeability is entered in dependence on the length stretching.

A unique identifier of the flow material containing elastomeric meltblown fibers is that the material remains water-impermeable even on strong stretching. It can thus be found that, on a stretching by 150%, 90% of the original watertighness still remains maintained. A standard SMMS product cannot be lengthened by 150% (cf. A in FIG. 4 and in Table 7) and even the SMMS material, which is stretched under heat treatment and contains conventional meltblown fibers, shows a fall in the water impermeability to 70% in relation to the initial value (cf. product B in FIG. 4 and in Table 7).

TABLE 7
Remaining water impermeability (%)

| | Length stretching of the material | | | | |
|---|---|---|---|---|---|
| Material | 0% | 10% | 50% | 100% | 150% |
| Product A: PP meltblown | 100 | 97.5% | 59.9% | — | — |
| Product B: PP meltblown | 100 | 100% | 100% | 96.9% | 69.2% |
| Product H: elastomeric | 100 | 100% | 100% | 95.4% | 91.2% |
| meltblown | | | | | |

It can be found in summary that, in relation to the prior art as it is shown by the products A and B in Table 7 or FIG. 4, the new nonwoven materials having the elastomeric meltblown fibers (product H) differ in that they even maintain a very good barrier property on a stretching by 150%.

The materials having the integrated elastomers in the meltblown layer show very good recovery properties. In the FIGS. 5 and 6, printouts of the tests of two materials are shown. Both products were stretched three times by 100%, with the permanent material lengthening being able to be read off the x axis. In FIG. 5, the product B having 55 g/m² basis weight and consisting of an SMMS material having conventional meltblown fibers has been tested. In FIG. 6, in contrast, an SMMS material having elastomeric meltblown fibers and having a basis weight of 50 g/m² has been tested. Here, too, the material was stretched three times by 100%. A comparison of the two materials shows that the recovery properties of the nonwoven material having the elastomeric meltblown fibers (FIG. 6) are substantially better than those of the nonwoven material not containing any elastic meltblown fibers.

1. A nonwoven material having elastic properties aligned in one direction, comprising:
either one multilayer composite including at least one layer in which fibers or filaments made of an elastic polymer are contained,
or of a homogeneous fiber and filament mix in which a portion of the fibers is composed of an elastic polymer,
wherein each case a larger part of the fibers or filaments is aligned in a direction under the application of heat which extends transversely to the direction in which the nonwoven material is elastic.

2. A nonwoven material in accordance with claim 1, wherein the multilayer composite contains elastic meltblown fibers and spunbond fibers.

3. A nonwoven material in accordance with claim 1, wherein the elastic meltdown fibers contain bicomponent fibers having an elastic portion.

4. A nonwoven material in accordance with claim 1, wherein the spunbound fibers are not elastic.

5. A nonwoven material in accordance with claim 1, wherein the homogeneous fiber mix is composed of a needle felt and/or a spunlaced product to which elastic fibers have been added.

6. A nonwoven material in accordance with claim 1, wherein a homogeneous fiber mix of a needle felt and/or a spunlaced product is combined with at least one layer of elastic meltdown fibers and/or spunbound fibers.

7. A nonwoven material in accordance with claim 1, wherein the needle felt and the spunlaced product also include viscose or natural fibers such as cellulose in addition to synthetic fibers.

8. A nonwoven material in accordance with claim 1, wherein one or more meltdown layers (M) are arranged between one or more spunbound layers (S), such as in the order SM, SMS, SMMS, SSMMS, SSMMSS, with the elastomeric layers being contained at least in one meltdown layer.

9. A nonwoven material in accordance with claim 1, wherein the elastic nonwoven layer is a liquid barrier layer or a particle retention layer.

10. A nonwoven material in accordance with claim 9, wherein the property as a liquid barrier layer or a particle retention layer also remains maintained after drawing or stretching the nonwoven material.

11. A nonwoven material in accordance with claim 1, wherein the product stretchability amounts to 0-700%, preferably to 50-400%.

12. A nonwoven material in accordance with claim 1, wherein the recovery (recovery property) of the product amounts to at least 60%, preferably to at least 80%, on a two-fold stretching by 100%.

13. A nonwoven material in accordance with claim 1, wherein the recovery (contracting property) of the product amounts to at least 50%, preferably to at least 70%, on a two fold stretching by 150%.

14. A nonwoven material in accordance with claim 1, wherein it is breathable.

15. A nonwoven material in accordance with claim 1, wherein it is hydrophilic.

16. A nonwoven material in accordance with claim 1, wherein a polymer having elastic properties is used as the meltdown fiber and having similar flow properties (with respect to the rheological and viscosity properties) as polypropylene.

17. A nonwoven material in accordance with claim 16, wherein it can be manufactured in an industrial production plant with high productivity.

18. A nonwoven material in accordance with claim 17, wherein the meltdown fibers are composed of the following mixture: more than 60% by weight of a triblock copolymer consisting of 70% by weight of styrene-ethylene/butylene-styrene and 30% by weight of styrene-ethylene/butylene-styrene, where the polystyrene portion of the polymer is 14% by weight (e.g. Kraton G®), 5-35% by weight of polypropylene suitable for processing in the meltblown method and an anti-blocking agent to improve the flow properties.

19. A nonwoven material in accordance with claim 18, wherein the meltblown fibers are composed of an elastic polyolefine, for example of a mettallocene-catalyzed copolymer of the polyethylene and/or polypropylene.

20. A nonwoven material in accordance with claim 18, wherein the meltdown fibers is composed of a thermoplastic elastic polyurethane.

21. A nonwoven material in accordance with claim 1, wherein, with a multilayer design, in addition to at least one meltblown layer having elastic fibers, spunbond layers made of one of the following materials are present: of polyolefine or polyester, or bicomponent polymer based on polypropylene and polyethylene, or of a polypropylene or polyester mixed with a bicomponent polypropylene/polyethylene or of an elastic polymer such as polyurethane, polystyrene block copolymer or an elastic polypropylene and/or polypropylene.

22. A nonwoven material in accordance with claim 21, wherein the spunbond layers and/or meltblown layers have a different design.

23. A nonwoven material in accordance with claim 1, wherein the layers of the multilayer design are bonded to one another by needlepunching, spunlacing, by thermobonding, by calendering with smooth rolls and/or engraved rolls and/or by infrared bonding.

24. A nonwoven material in accordance with claim 1, wherein the basis weight of the multilayer design amounts to 7 g/m² up to 400 g/m², where the elastic meltdown layers amount to 1 to 60% by weight.

25. A nonwoven material in accordance with claim 1, wherein the basis weight of the needle nonwoven/spunlaced product or needle nonwoven together with elastic meltblown layers amounts to 40-700 g/m², where the elastic meltblown layers amount to 1 to 60% by weight.

26. A nonwoven material in accordance with claim 1, wherein the meltblown layer provided with elastic properties has a fiber thickness of 0.01 to 1.2 denier, preferably 0.01 to 0.5 denier.

27. A method of manufacturing a nonwoven material in accordance with claim 1 to 26, characterized in that the prefabricated nonwoven material web is drawn either in the running direction or transversely to the running direction for the aligning of the fibers/filaments under the application of heat.

28. A method in accordance with claim 27, wherein the transport speed in the longitudinal direction, measured in %, is lowered by more than the width increase in % to generate the elastic properties of the nonwoven material in the longitudinal direction and the increase of the basis weight belonging thereto.

29. A method in accordance with claim 27, wherein the width restriction, measured in %, is higher than the increase of the transport speed in the longitudinal direction, measured in %, to generate elastic properties of the nonwoven material in the transverse direction and the increase of the basis weight belonging thereto.

30. An apparatus for the carrying out of the method in accordance with claim 1, characterized in that it has an oven and at least one drawing device to draw the nonwoven material web.

31. An apparatus in accordance with claim 30, wherein the drawing device to draw the nonwoven material web in the direction transverse to its transport direction has two wheel-shaped gripping apparatuses arranged to the side of the nonwoven material web and having receiving regions arranged at its periphery to grip the nonwoven material web.

32. An apparatus in accordance with claim 1, wherein the drawing device to draw the nonwoven material web in the direction longitudinal to its transport direction is composed of at least two rolls via which the nonwoven material web is fixed by friction, wherein it is pulled at a speed higher in comparison with the entry speed of the nonwoven material web into the oven so that the nonwoven material web is drawn in the longitudinal direction.

33. An apparatus in accordance with claim 1, wherein the drawing device to draw the nonwoven material web in the direction longitudinal to its transport direction is composed of at least two oppositely disposed rolls between which the nonwoven material web is clamped and which are derivable at a higher peripheral speed than entry speed of the nonwoven material web into the oven so that the drawing of the nonwoven material web in the longitudinal direction takes place.

34. An apparatus in accordance with claim 1, wherein a temperature is set in the oven between the softening point and the melting point of the respectively processed thermoplastic fibers.

35. An apparatus in accordance with claim 1, wherein the drawing/pulling speed of the nonwoven material web amounts to 5-150 m/min, preferably to 40-100 m/min, on the drawing into the width.

36. An apparatus in accordance with claim 1, wherein the processing speed of the nonwoven material web amounts to 5-400 m/min, preferably to 80-250 m/min, on the drawing in the longitudinal direction.
